(19) United States
(12) Patent Application Publication
(10) Pub. No.: US 2021/0121084 A1
(43) Pub. Date: Apr. 29, 2021

(54) METHOD AND APPARATUS FOR MEASURING BLOOD PRESSURE USING SKIN IMAGES

(71) Applicant: RESEARCH & BUSINESS FOUNDATION SUNGYUNKWAN UNIVERSITY, Suwon-si (KR)

(72) Inventors: Kwang Seok HONG, Suwon-si (KR); Jin Soo PARK, Suwon-si (KR); Sang Il JO, Icheon-si (KR)

(73) Assignee: RESEARCH & BUSINESS FOUNDATION SUNGKYUNKWAN UNIVERSITY, Suwon-si (KR)

(21) Appl. No.: 17/077,012

(22) Filed: Oct. 22, 2020

(30) Foreign Application Priority Data
Oct. 24, 2019 (KR) 10-2019-0132789

(51) Int. Cl. A61B 5/021; A61B 5/022; A61B 5/00; A61B 5/103

(52) U.S. Cl. CPC A61B 5/02125; A61B 5/022; A61B 5/7257; A61B 5/1032; A61B 5/441

(57) ABSTRACT

A blood pressure measurement method, includes: receiving an image comprising a skin image of a user; converting each color data of skin regions of interest in a predetermined part of the skin image into frequency domain data; calculating a maximum peak value of pulses in pulse related frequency domain data of the frequency domain data, a maximum frequency value corresponding to the maximum peak value, and a maximum peak frequency phase difference of a phase difference of the maximum frequency value between the skin regions; calculating a pulse transit time as a function of the maximum frequency value and the maximum peak frequency phase difference; and calculating a blood pressure of the user as a function of the maximum peak value, the maximum frequency value, and the pulse transit time.

### CROSS-REFERENCE TO RELATED APPLICATIONS

This application claims the benefit under 35 USC 119(a) of Korean Patent Application No. 10-2019-0132789 filed on Oct. 24, 2019, in the Korean Intellectual Property Office, the entire disclosure of which is incorporated herein by reference for all purposes.

### BACKGROUND

### 1. Field

The following description disclosure relates to a method and an apparatus for measuring a blood pressure using skin images.

### 2. Description of Related Art

Recently, the interest of the World Health Organization (WHO) on chronic diseases (for example, high blood pressure) is increasing and the WHO has selected the “high blood pressure” as the theme of the 2013 world health day. Countries around the world are urging efforts to prevent high blood pressure and manage blood pressure in daily life. In Korea, as the interest in cardio-cerebrovascular disease is increasing, the Centers for Disease Control and Prevention (CDC) celebrated World High Blood Pressure Day on May 17, 2015 by grasping the current state of prevention and management of high blood pressure and recommending guidelines for healthy life. The high blood pressure is the most common and powerful risk factor for cardiovascular diseases and if the high blood pressure is not managed, it may cause stroke and myocardial infarction. Therefore, it is necessary to check and manage a level by regularly measuring a blood pressure. For this reason, in recent years, technologies of measuring and analyzing an individual's blood pressure state are attracting attention.

However, according to a blood pressure measuring method using an existing sphygmomanometer, a guff connected to a device is strongly attached to a wrist to measure a blood pressure and for accurate measurement, a subject who measures a blood pressure needs to roll up the sleeves. Further, the subject who measures a blood pressure feels a pain due to a pressure applied from the guff.

In order to solve this problem, in recent years, technologies which measure a biosignal using a contact type biosignal (PPG or ECG) measurement device and calculate a blood pressure of a user by time-serially analyzing a frequency of the signal have been developed. Studies on a blood pressure measurement technology using a contact type biosignal measurement equipment of the related art are basically performed by a detecting device which measures a biosignal and a display device which shows the biosignal to users. This technology acquires data by irradiating light to the capillary and converting an absorbed and reflected amount of the light into a signal using equipment in which an IR light source sensor and a light receiving sensor are mounted. However, the technology has a disadvantage in that additional equipment is used to be in direct contact with a skin of a user.

According to the study on a blood pressure measurement technology using a biosignal of a face skin image of the related art, a pulse transit time (PTT) is calculated using a distance change of two pulse signals calculated from two different regions of interest of a face image and the blood pressure is measured using the calculated PTT. However, when the pulse transit time is calculated using the distance change of two different pulse signals calculated from the face image, a large measurement error may be obtained.

Accordingly, there is a necessity for a method and an apparatus for measuring a blood pressure with high accuracy through a skin image in a contactless manner using a normal camera, an IR camera, and a smartphone which are possessed by a user without using additional equipment.

### SUMMARY

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

In one general aspect, a blood pressure measurement method, includes: receiving an image comprising a skin image of a user; converting each color data of skin regions of interest in a predetermined part of the skin image into frequency domain data; calculating a maximum peak value of pulses in pulse related frequency domain data of the frequency domain data, a maximum frequency value corresponding to the maximum peak value, and a maximum peak frequency phase difference of a phase difference of the maximum frequency value between the skin regions; calculating a pulse transit time as a function of the maximum frequency value and the maximum peak frequency phase difference; and calculating a blood pressure of the user as a function of the maximum peak value, the maximum frequency value, and the pulse transit time.

The calculating of the blood pressure may include receiving a height and a weight of the user, and calculating the blood pressure of the user as a function of the maximum peak value, the maximum frequency value, the pulse transit time, the height, and the weight.

The predetermined part may include a plurality of predetermined parts, and the method may further include: converting the color data into the frequency domain data for each of the plurality of parts, calculating the maximum peak frequency phase difference for the frequency domain data for each of the plurality of parts, and calculating the blood pressure for the maximum peak frequency phase difference for the frequency domain data for each of the plurality of parts, and averaging the calculated blood pressure for the plurality of parts.

The method may further include calculating a modified blood pressure of the user using a blood pressure regression analysis equation based on a plurality of first blood pressure data of the calculated blood pressure and a plurality of second blood pressure data measured by a sphygmomanometer.

The converting of the color data into the frequency domain data may include changing a color system of a plurality of pixels of the skin image corresponding to each of the skin regions from an RGB color system to YC_{g}Cₒ and YC_{b}Cᵣ color systems; calculating a weighted average value of C_{g} and C_{b} color data included in the YC_{g}Cₒ and YC_{b}Cᵣ color systems; and applying Fast Fourier Transform (FFT) to the weighted average value of the C_{g} and C_{b} color data.

In another general aspect, a blood pressure measurement apparatus, includes an interface configured to receive a captured image including a skin image of a user and one or more processors. The one or more processors are configured to convert each color data of skin regions of interest in a predetermined part of the skin image into frequency domain data, calculate a maximum peak value of pulses in the frequency domain data, a maximum frequency value corresponding to the maximum peak value, and a maximum peak frequency phase difference of a phase difference of the maximum frequency value between the skin regions, calculate a pulse transit time as a function of the maximum frequency value and the maximum peak frequency phase difference, and calculate the blood pressure of the user as a function of the maximum peak value, the maximum frequency value, and the pulse transit time.

When a height and a weight of the user are further received through the interface, the one or more processors may be further configured to calculate the blood pressure of the user as a function of the maximum peak value, the maximum frequency value, the pulse transit time, the height, and the weight.

When there is a plurality of predetermined parts of the skin image, the one or more processors may be configured to calculate the blood pressure for each of the plurality of parts, and an average blood pressure of the blood pressure for each of the plurality of parts.

The one or more processors may be configured to further calculate a modified blood pressure of the user using a blood pressure regression analysis equation based on a plurality of first blood pressure data of the calculated blood pressure and a plurality of second blood pressure data measured by a sphygmomanometer.

The one or more processors may be configured to change a color system of a plurality of pixels of the plurality of images included in the image corresponding to the skin regions from an RGB color system to YC_{g}Cₒ and YC_{b}Cᵣ color systems, calculate a weighted average value of C_{g} and C_{b} color data included in the YC_{g}Cₒ and YC_{b}Cᵣ color systems, and apply Fast Fourier Transform (FFT) to the weighted average value of the C_{g} and C_{b} color data.

In another general aspect, a blood pressure measurement method, includes receiving an image comprising a skin image of a user, converting color data of skin regions of interest in one or more predetermined parts of the skin image into corresponding frequency domain data, calculating maximum peak values of pulses in the corresponding frequency domain data, maximum frequency values corresponding to the maximum peak values, and maximum peak frequency phase differences of the maximum frequency values, calculating pulse transit times of corresponding ones of the maximum frequency values and the maximum peak frequency phase differences, and calculating blood pressures as a function of corresponding ones of the maximum peak values, the maximum frequency values, and the pulse transit times.

An average blood pressure of the blood pressures may be indicated as a blood pressure of the user.

The function of calculating of the blood pressure may further comprise a height and a weight of the user.

Other features and aspects will be apparent from the following detailed description, the drawings, and the claims.

### BRIEF DESCRIPTION OF DRAWINGS

FIG. 1 is a flowchart illustrating a blood pressure measurement method using a skin image according to one or more embodiments of the present disclosure.

FIG. 2 is a flowchart explaining a conversion method to frequency domain data according to one or more embodiments of the present disclosure.

FIG. 3 is a block diagram of a blood pressure measurement apparatus using a skin image according to one or more embodiments of the present disclosure.

FIG. 4 is a diagram illustrating a process of generating frequency domain data according to one or more embodiments of the present disclosure.

FIG. 5 is a diagram explaining a method of calculating a blood pressure for a plurality of parts according to one or more embodiments of the present disclosure.

FIG. 6 is a diagram for explaining a method of calculating a blood pressure regression analysis equation DB according to one or more embodiments of the present disclosure.

Throughout the drawings and the detailed description, the same reference numerals refer to the same elements. The drawings may not be to scale, and the relative size, proportions, and depiction of elements in the drawings may be exaggerated for clarity, illustration, and convenience.

### DETAILED DESCRIPTION

“The following detailed description is provided to assist the reader in gaining a comprehensive understanding of the methods, apparatuses, and/or systems described herein. However, various changes, modifications, and equivalents of the methods, apparatuses, and/or systems described herein will be apparent after an understanding of the disclosure of this application. For example, the sequences of operations described herein are merely examples, and are not limited to those set forth herein, but may be changed as will be apparent after an understanding of the disclosure of this application, with the exception of operations necessarily occurring in a certain order. Also, descriptions of features that are known after understanding of the disclosure of this application may be omitted for increased clarity and conciseness.

The features described herein may be embodied in different forms, and are not to be construed as being limited to the examples described herein. Rather, the examples described herein have been provided merely to illustrate some of the many possible ways of implementing the methods, apparatuses, and/or systems described herein that will be apparent after an understanding of the disclosure of this application.

Throughout the specification, when an element, such as a layer, region, or substrate, is described as being “on,” “connected to,” or “coupled to” another element, it may be directly “on,” “connected to,” or “coupled to” the other element, or there may be one or more other elements intervening therebetween. In contrast, when an element is described as being “directly on,” “directly connected to,” or “directly coupled to” another element, there can be no other elements intervening therebetween.

As used herein, the term “and/or” includes any one and any combination of any two or more of the associated listed items.

Although terms such as “first,” “second,” and “third” may be used herein to describe various members, components, regions, layers, or sections, these members, components, regions, layers, or sections are not to be limited by these terms. Rather, these terms are only used to distinguish one member, component, region, layer, or section from another member, component, region, layer, or section. Thus, a first member, component, region, layer, or section referred to in examples described herein may also be referred to as a second member, component, region, layer, or section without departing from the teachings of the examples.

Spatially relative terms such as “above,” “upper,” “below,” and “lower” may be used herein for ease of description to describe one element's relationship to another element as shown in the figures. Such spatially relative terms are intended to encompass different orientations of the device in use or operation in addition to the orientation depicted in the figures. For example, if the device in the figures is turned over, an element described as being “above” or “upper” relative to another element will then be “below” or “lower” relative to the other element. Thus, the term “above” encompasses both the above and below orientations depending on the spatial orientation of the device. The device may also be oriented in other ways (for example, rotated 90 degrees or at other orientations), and the spatially relative terms used herein are to be interpreted accordingly.

The terminology used herein is for describing various examples only, and is not to be used to limit the disclosure. The articles “a,” “an,” and “the” are intended to include the plural forms as well, unless the context clearly indicates otherwise. The terms “comprises,” “includes,” and “has” specify the presence of stated features, numbers, operations, members, elements, and/or combinations thereof, but do not preclude the presence or addition of one or more other features, numbers, operations, members, elements, and/or combinations thereof.

The features of the examples described herein may be combined in various ways as will be apparent after an understanding of the disclosure of this application. Further, although the examples described herein have a variety of configurations, other configurations are possible as will be apparent after an understanding of the disclosure of this application.

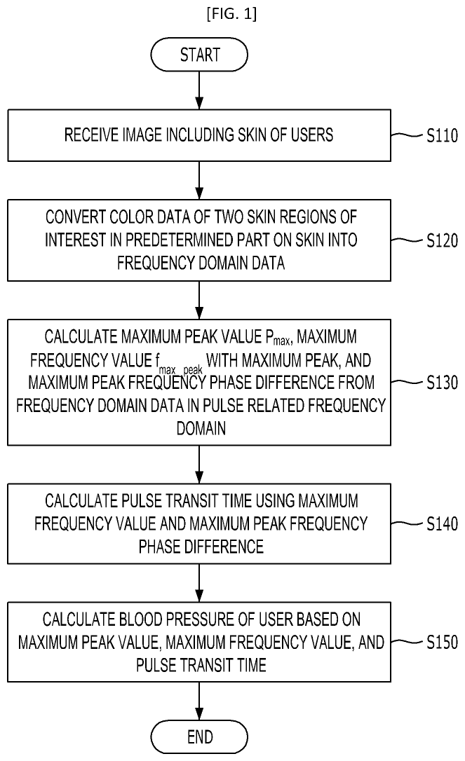

FIG. 1 is a flowchart illustrating a blood pressure measurement method using a skin image according to one or more embodiments of the present disclosure.

In operation S110, a blood pressure measurement apparatus receives an image including a skin of a user.

Here, the blood pressure measurement apparatus may receive an image including a skin of a user captured using a camera included therein or an external general camera an IR camera, or the like. In one example, the image including the skin of the user may refer to a moving image in which a face, a wrist, an arm, or the like of the user continuously appears at the same location or continuous photographs with a predetermined time interval. For example, when the blood pressure measurement apparatus is mounted in a smartphone, an image obtained by capturing a skin of a user using the smartphone may also be received.

In addition, the blood pressure measurement apparatus may perform a pre-processing task to detect the captured body part of the user, such as a face, a wrist, or an arm or detect the captured skin color.

In operation S120, the blood pressure measurement apparatus converts each color data of two skin regions of interest in a predetermined part on the skin included in the image into frequency domain data.

In one example, the predetermined part on the skin may be a region on the skin having an arbitrary shape, for example, may be a rectangular shape or a circular shape. Further, two skin regions of interest may be two points included in the corresponding part.

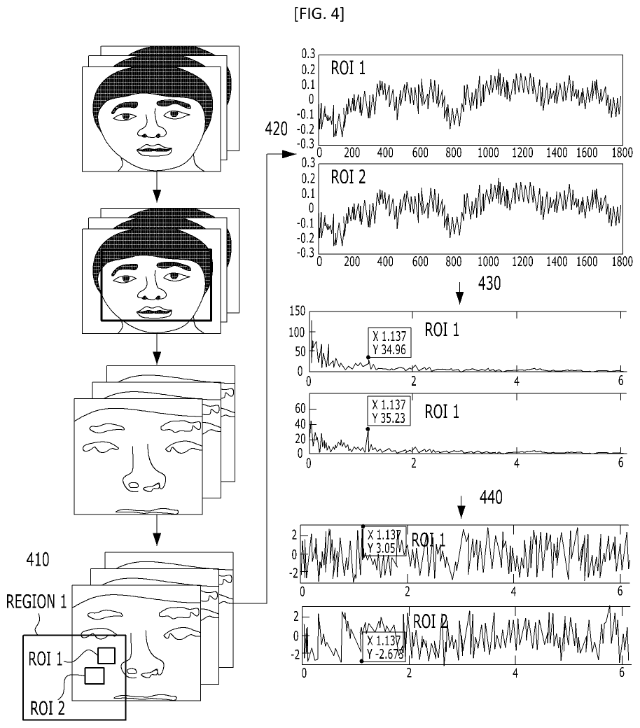

For example, in FIG. 4, the blood pressure measurement apparatus generates time-series data (420) by time-serially arranging a plurality of color data average values corresponding to skin regions (410) ROI1 and RIO2 of interest for two different skin regions of interest and then converts two different time series data into frequency domain data (430).

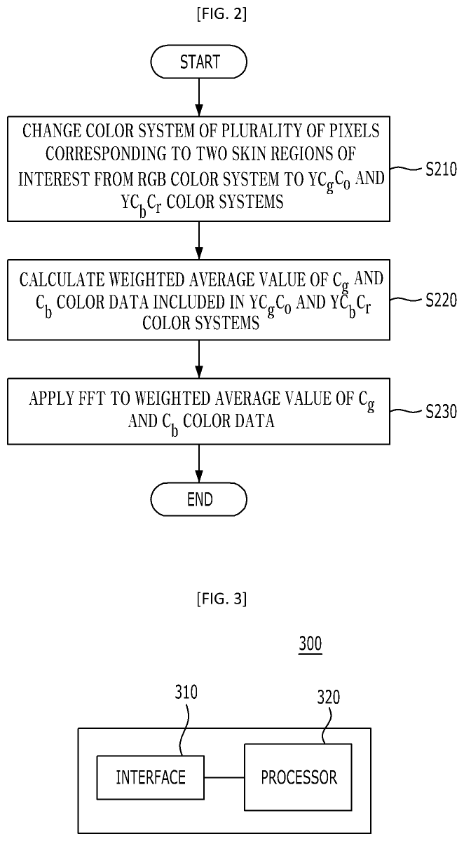

In one example, a detailed method of converting color data into frequency domain data by the blood pressure measurement apparatus will be described in detail in the description of FIG. 2.

In operation S130, the blood pressure measurement apparatus calculates a maximum peak value Pₘₐₓ with a largest magnitude in a pulse related frequency domain, a maximum frequency value f_{max_peak} corresponding to the maximum peak value, and a maximum peak frequency phase difference which indicates a phase difference of the maximum frequency values between two skin regions of interest.

In one example, the maximum peak value Pₘₐₓ is a peak value of the frequency domain data with the largest magnitude and the maximum frequency value f_{max_peak} is a frequency value at which the magnitude is the maximum peak value Pₘₐₓ. The maximum peak frequency phase difference may be a difference of a phase (angle) of the maximum frequency value f_{max_peak} between two skin regions of interest.

For example, referring to FIG. 4, the maximum peak frequency phase difference may be calculated by the following Equation 1 after obtaining a phase value corresponding to the maximum frequency value f_{max_peak} between two skin regions of interest from a result 440 with a phase value for every frequency.

$${θ}_{d} = \{ \begin{matrix}( 2 π ) - \vert {θ}_{{ROI}_{1}} - {θ}_{{ROI}_{2}} \vert & if \vert {θ}_{{ROI}_{1}} - {θ}_{{ROI}_{2}} \vert > π \\ \vert {θ}_{{ROI}_{1}} - {θ}_{{ROI}_{2}} \vert & otherwise\end{matrix} \qquad Equation 1$$

Here, θ_{d} is a phase difference between Iwo skin regions or interest and θ_{ROI1}, and θ_{ROI2} are phases of the maximum frequency values with the maximum peak in a first skin region of interest and a second skin region of interest, respectively.

Desirably, the blood pressure measurement apparatus may limit a region of the frequency domain where the maximum peak value Pₘₐₓ, the maximum frequency value f_{max_peak}, and the maximum peak frequency phase difference are calculated to 0.65 Hz to 4 Hz. This is because in a normal condition, pulses per minute may be measured from approximately 40 to 240 depending on a level of stability or excitement.

In operation S140, the blood pressure measurement apparatus may calculate a pulse transit time using the maximum frequency value and the maximum peak frequency phase difference.

For example, the pulse transit time may be calculated by Equation 2.

$$P T T ( Pulse Transit Time ) = \{ \begin{matrix}\frac{( 2 π - {θ}_{d} )}{2 π} \times {f}_{max\_peak} & if {θ}_{d} > π \\ \frac{{θ}_{d}}{2 π} \times {f}_{max\_peak} & otherwise\end{matrix} \qquad Equation 2$$

Here, PTT is a pulse transit time, θ_{d} is a maximum peak frequency phase difference, and f_{max_peak} is a maximum frequency value with a maximum peak.

Finally, in operation S150, the blood pressure measurement apparatus calculates a blood pressure of a user based on the pulse transit time, the maximum peak value, and the maximum frequency value.

That is, the blood pressure measurement apparatus may calculate the blood pressure of a user based on the pulse transit time, the maximum peak value, and the maximum frequency value after calculating the pulse transit time using the maximum frequency value and the maximum peak frequency phase difference.

According to another embodiment, the blood pressure measurement apparatus may further receive a height and a weight of the user prior to operation S150 and when the blood pressure of the user is calculated, the blood pressure measurement apparatus may calculate the blood pressure of the user further based on the height and the weight.

That is, the blood pressure measurement apparatus may further receive information on a height and a weight of a user to calculate the blood pressure of the user using the information.

For example, the blood pressure measurement apparatus may calculate the blood pressure of the user using the following Equation 3.

BP_{S}=PTT×α₁+f_{max_peak}×α₂+Pₘₐₓ×α₃+H×α₄+W×α₅+α₆

BP_{D}=PTT×β₁+f_{max_peak}×β₂+Pₘₐₓ×β₃+H×β₄+W×β₅+β₆ Equation 3:

Here, BP_{S} is a systolic blood pressure, BP_{D} is a diastolic blood pressure, PTT is a pulse transit time, f_{max_peak} is a maximum frequency value with a maximum peak, Pₘₐₓ is a maximum peak value, H is a height of the user, and W is a weight of the user. Further, α₁ to α₆ and β₁ to β₆ are coefficients which vary depending on the used DB.

According to still another embodiment, when a plurality of predetermined parts on the skin is provided and operations S120, S130, S140, and S150 are performed in each of the plurality of parts, an average blood pressure of the user may be calculated from an average value of the blood pressures of the plurality of parts.

That is, the blood pressure measurement apparatus may calculate a blood pressure using two skin regions of interest for each of the plurality of parts of one image. Further, the blood pressure measurement apparatus may calculate an average blood pressure of the user using an average value of the blood pressures calculated for the plurality of parts.

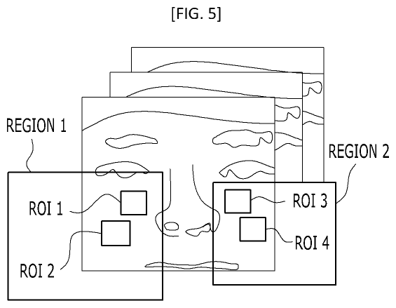

For example, referring to FIG. 5, the blood pressure measurement apparatus may calculate a blood pressure for a region 1 located on a right cheek of the user and a region 2 located on a left cheek and then calculate an average blood pressure using an average value thereof.

By doing this, the blood pressure measurement apparatus may stably measure a blood pressure of the user more robustly from a measurement error due to the difference in the illumination and the like.

According to still another embodiment, the blood pressure measurement apparatus may calculate a modified blood pressure which is a blood pressure of the user more robustly modified, using a blood pressure regression analysis equation DB based on a plurality of first blood pressure data which is the calculated blood pressure data of the user and a plurality of second blood pressure data which is blood pressure data measured by a sphygmomanometer.

In the meantime, a result obtained by comparing a blood pressure measurement result (represented as “image”) by the blood pressure measurement method using a skin image according to the embodiment of the present disclosure and a blood pressure measurement result (represented as “sphygmomanometer”) using a sphygmomanometer is as represented in the following Table 1.

TABLE 1

| | | First | | Second | | Third | |
|---|---|---|---|---|---|---|---|
| Classification | | Systole | Diastole | Systole | Diastole | Systole | Diastole |
| Subject 1 | Image | 134 | 81 | 137 | 72 | 136 | 82 |
| | Sphygmomanometer | 137 | 78 | 134 | 73 | 135 | 74 |
| Subject 2 | Image | 123 | 74 | 118 | 71 | 112 | 67 |
| | Sphygmomanometer | 120 | 75 | 119 | 66 | 118 | 69 |
| Subject 3 | Image | 134 | 70 | 133 | 80 | 119 | 72 |
| | Sphygmomanometer | 139 | 71 | 132 | 75 | 118 | 67 |
| Subject 4 | Image | 117 | 70 | 124 | 74 | 119 | 72 |
| | Sphygmomanometer | 112 | 65 | 126 | 65 | 118 | 67 |
| Subject 5 | Image | 110 | 77 | 114 | 69 | 113 | 75 |
| | Sphygmomanometer | 108 | 73 | 110 | 66 | 108 | 70 |
| Subject 6 | Image | 109 | 67 | 118 | 73 | 115 | 70 |
| | Sphygmomanometer | 113 | 71 | 115 | 72 | 112 | 73 |

At this time, a biosignal was measured using a face image and a sphygmomanometer for 15 seconds per one experiment and photographing was performed at 30 frames per second. A total of six subjects (four males and two females) participated and the face image and the blood pressure of each subject were measured three times, that is, a total of 18 times was measured to calculate the blood pressure.

Further, a result obtained by measuring an error rate in Table 1 is represented in the following Table 2.

TABLE 2

Blood pressure

| Classification | Systole | Diastole |
|---|---|---|
| Error rate | 2.48% | 5.23% |

As described above, the blood pressure measurement method using a skin image according to one or more embodiments of the present disclosure may extract a biosignal using a skin image of a user and more accurately and stably measure the blood pressure using this signal.

FIG. 2 is a flowchart for explaining a conversion method to frequency domain data according to one or more embodiments of the present disclosure.

In operation S210, the blood pressure measurement apparatus changes a color system of a plurality of pixels corresponding to two skin regions of interest from a plurality of images included in the image from an RGB color system to YC_{g}Cₒ and YC_{b}Cᵣ color systems.

In one example, the blood pressure measurement apparatus may change the plurality of images having the RGB color system into YC_{g}Cₒ and YC_{b}Cᵣ color systems, respectively. In one example, the YC_{g}Cₒ color system is a color space configured by a luminance Y, a green color difference C_{g}, and an orange color difference Cₒ and the YC_{b}Cᵣ color system is a color space configured by a luminance Y, a blue color difference C_{b}, and a red color difference Cᵣ.

In the meantime, the blood pressure measurement apparatus may convert the RGB color system into another color system. For example, a computer device may convert the RGB color system into various color systems such as YUV, HSV, YC_{b}Cᵣ, or YC_{g}Cₒ. In one example, the color data may use one of color difference components which are less affected by the surrounding environments (for example, illumination, etc.). For example, at least one of a C_{b} value and a Cᵣ value of the YC_{b}Cᵣ may be used. Further, at least one of a C_{g} value and a Cₒ value of the YC_{g}Cₒ may be used. Moreover, any one of two color difference components which is more robust to the change of the illumination may be used. For example, only C_{g} value of YC_{g}Cₒ may be used. In one example, the computer device may extract an average value of C_{g} color data of the skin region as color data. Moreover, the color data may be a value combined by applying a weight to at least one color component in various color systems such as RGB, YUV, HSV, YCbCᵣ, and YC_{g}Cₒ. When the color components are combined, the color data may be a value obtained by adding values to which different weights are applied depending on the color system and a type of color component. The face image having the RGB color system may be changed to the YC_{g}Cₒ color system and it is assumed that the C_{g} value of YC_{g}Cₒ is used hereinbelow. The C_{g} value may be referred to as a C_{g} signal.

In operation S220, the blood pressure measurement apparatus calculates a weighted average value of C_{g} and C_{b} color data included in the YC_{g}Cₒ and YC_{b}Cᵣ color systems.

That is, the blood pressure measurement apparatus may time-serially calculate the weighted average value of C_{g} color data of the YC_{g}Cₒ color system and C_{b} color data of the YC_{b}Cᵣ color system for the plurality of images.

Finally, in operation S230, the blood pressure measurement apparatus applies Fast Fourier Transform (FFT) to the weighted average value of C_{g} and C_{b} color data.

That is, the blood pressure measurement apparatus may calculate the FFT on the weighted average value of C_{g} and C_{b} color data which are time-serially calculated to convert the weighted average value into frequency domain data.

FIG. 3 is a block diagram of a blood pressure measurement apparatus using a skin image according to one or more embodiments of the present disclosure.

Referring to FIG. 3, a blood pressure measurement apparatus 300 using a skin image according to one or more embodiments of the present disclosure includes an interface 310 and a processor 320.

In one example, the blood pressure measurement apparatus 300 using a skin image according to one or more embodiments of the present disclosure may be mounted in smartphones, tablet PCs, wearable apparatuses, notebook PCs, desktop PCs, and the like.

In the meantime, when the blood pressure measurement apparatus 300 using a skin image is mounted in a device in which a camera is already mounted, such as a smartphone, the blood pressure measurement apparatus 300 using a skin image photographs a skin of the user using the camera of the smartphone to more conveniently measure the blood pressure of the user.

The interface 310 receives an image including the skin of the user. The interface 310 may be a connection terminal which wirely receives the image captured by the camera or a communication module which wirelessly receives the image.

The processor 320 converts each color data of two skin regions of interest in a predetermined part on the skin included in the image into frequency domain data.

According to another embodiment, the processor 320 may change a color system of a plurality of pixels corresponding to two skin regions of interest in the plurality of images included in the image from the RGB color system into the YC_{g}Cₒ and YC_{b}Cᵣ color systems, calculate a weighted average value of C_{g} and C_{b} color data included in the YC_{g}Cₒ and YC_{b}Cᵣ color systems, and apply the Fast Fourier Transform (FFT) to the weighted average value of C_{g} and C_{b} color data.

Finally, the processor 320 calculates a maximum peak value Pₘₐₓ with the largest magnitude, a maximum frequency value f_{max_peak} corresponding to the maximum peak value, and a maximum peak frequency phase difference indicating a phase difference of the maximum frequency value between two skin regions of interest, calculates a pulse transit time using the maximum frequency value and the maximum peak frequency phase difference, and calculates a blood pressure of the user based on the maximum peak value, the maximum frequency value, and the pulse transit time.

According to another embodiment, when the interface 310 receives the height and the weight of the user, the processor 320 may calculate the blood pressure of the user further based on the height and the weight.

According to still another embodiment, when there is a plurality of predetermined parts on the skin and the processor 320 calculates a blood pressure of the user for each of the plurality of parts, an average blood pressure may be further calculated from an average value of blood pressures of the plurality of parts.

According to still another embodiment, the processor 320 may further calculate a modified blood pressure which is a modified blood pressure of the user, using a blood pressure regression analysis equation DB based on a plurality of first blood pressure data which is the calculated blood pressure data of the user and a plurality of second blood pressure data which is blood pressure data measured by a sphygmomanometer and the calculated blood pressure of the user.

FIG. 4 is a view illustrating a process of generating a frequency phase of two different skin regions of interest according to one or more embodiments of the present disclosure.

After generating time-series data (420) by time-serially arranging a plurality of color data average values corresponding to skin regions (410) ROI1 and RIO2 of interest for two different skin regions of interest, the Fast Fourier Transform (FFT) is applied to the color data calculated from two different skin regions of interest to convert color data into frequency domain data (430).

Further, the maximum peak frequency phase value corresponding to a frequency between two different skin regions of interest is acquired from a result 440 in which a phase value for every frequency is represented and the phase difference of the maximum peak frequency may be calculated.

FIG. 5 is a diagram for explaining a method of calculating a blood pressure for a plurality of parts according to one or more embodiments of the present disclosure.

The blood pressure measurement apparatus calculates a blood pressure for a region 1 located on a right cheek of the user and a region 2 located on a left cheek and then may calculate an average blood pressure using an average value thereof. By doing this, the blood pressure measurement apparatus may stably measure a blood pressure of the user more robustly from a measurement error due to the difference in the illumination and the like.

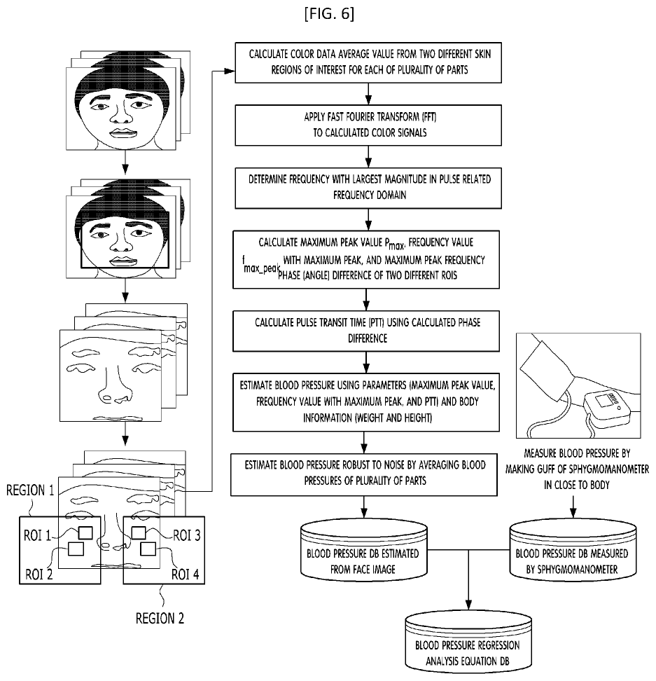

FIG. 6 is a diagram for explaining a method of calculating a blood pressure regression analysis equation DB according to one or more embodiments of the present disclosure.

As an example, the blood pressure is measured by photographing a face image using a camera and also using a sphygmomanometer. Two different skin regions (a left cheek and a right cheek) of interest for each of the plurality of parts are detected from the captured face image. The RGB color system of the detected skin region of interest as an example of various color systems is converted into the YC_{g}Cₒ color system to calculate C_{g} and C_{b} color data weighted average value to which a weight is applied. The Fast Fourier Transform (FFT) is applied to the calculated color data weighted average value and a frequency with a largest magnitude in a pulse-related frequency domain (0.65 to 4 Hz) is selected.

In order to measure a blood pressure, parameters (a maximum peak value Pₘₐₓ, a frequency value f_{max_peak} with a maximum peak, and a maximum peak frequency phase (angle) difference of two different ROIs) are calculated and a pulse transit time (PTT) is calculated using the frequency value with a maximum peak and the phase difference. The blood pressure is estimated using the calculated parameters and body information (a weight and a height). The estimated blood pressures of the plurality of parts are averaged to measure a robust blood pressure using a skin image and the measured blood pressure is stored in “a blood pressure DB estimated from a face image”. The blood pressure measured using the sphygmomanometer is stored in “a blood pressure DB measured by a sphygmomanometer”. The regression analysis is applied to two DBs to calculate a regression line (or curve) equation and the regression line equation is stored in a “blood pressure regression analysis equation DB”.

A straight line obtained by representing a point set on a scatter diagram with a straight line represents a relationship between two variables and in the present disclosure, the regression straight line equation is derived using the “blood pressure DB estimated from the face image” and the “blood pressure DB measured using a sphygmomanometer”. The regression straight line equation is represented by Equation 4.

y=ax+b Equation 4:

Here, y indicates a modified blood pressure and x indicates a blood pressure value estimated from a face image. In a result obtained by applying actual data, constants a and b may vary depending on used data.

A regression straight line equation calculated by applying the regression analysis to the “blood pressure DB estimated from the face image” and the “blood pressure DB calculated by a sphygmomanometer” is represented by the following Equation 5. The regression straight line equation may slightly vary depending on a DB used for calculation.

BP_{S}=1.0459x−6.1793

BP_{D}=0.5313x−31.938 Equation 5:

A curve obtained by representing a point set on a scatter diagram with a curve represents a relationship between two variables and in the present disclosure, the regression curve equation is derived using the “blood pressure DB estimated from the face image” and the “blood pressure DB measured using a sphygmomanometer”. The regression curve equation is represented by Equation 6.

y=ax²+bx+c Equation 6:

Here, y indicates a modified blood pressure and x indicates a blood pressure value estimated from a face image. In a result obtained by applying actual data, constants a, b, and c may vary depending on used data.

A regression curve equation calculated by applying the regression analysis to the “blood pressure DB estimated from the face image” and the “blood pressure DB calculated by a sphygmomanometer” is represented by the following Equation 7. The regression curve equation may slightly vary depending on a DB used for calculation.

BP_{S}=0.0117x²−1.8405x+171.25

BP_{D}=0.0331x²−4.4011x+215.05 Equation 7:

The blood pressure measurement apparatus, blood pressure measurement apparatus 300, interface 310, and processor 320 in FIGS. 1-6 that perform the operations described in this application are implemented by hardware components configured to perform the operations described in this application that are performed by the hardware components. Examples of hardware components that may be used to perform the operations described in this application where appropriate include controllers, sensors, generators, drivers, memories, comparators, arithmetic logic units, adders, subtractors, multipliers, dividers, integrators, and any other electronic components configured to perform the operations described in this application. In other examples, one or more of the hardware components that perform the operations described in this application are implemented by computing hardware, for example, by one or more processors or computers. A processor or computer may be implemented by one or more processing elements, such as an array of logic gates, a controller and an arithmetic logic unit, a digital signal processor, a microcomputer, a programmable logic controller, a field-programmable gate array, a programmable logic array, a microprocessor, or any other device or combination of devices that is configured to respond to and execute instructions in a defined manner to achieve a desired result. In one example, a processor or computer includes, or is connected to, one or more memories storing instructions or software that are executed by the processor or computer. Hardware components implemented by a processor or computer may execute instructions or software, such as an operating system (OS) and one or more software applications that run on the OS, to perform the operations described in this application. The hardware components may also access, manipulate, process, create, and store data in response to execution of the instructions or software. For simplicity, the singular term “processor” or “computer” may be used in the description of the examples described in this application, but in other examples multiple processors or computers may be used, or a processor or computer may include multiple processing elements, or multiple types of processing elements, or both. For example, a single hardware component or two or more hardware components may be implemented by a single processor, or two or more processors, or a processor and a controller. One or more hardware components may be implemented by one or more processors, or a processor and a controller, and one or more other hardware components may be implemented by one or more other processors, or another processor and another controller. One or more processors, or a processor and a controller, may implement a single hardware component, or two or more hardware components. A hardware component may have any one or more of different processing configurations, examples of which include a single processor, independent processors, parallel processors, single-instruction single-data (SISD) multiprocessing, single-instruction multiple-data (SIMD) multiprocessing, multiple-instruction single-data (MISD) multiprocessing, and multiple-instruction multiple-data (MIMD) multiprocessing.

The methods illustrated in FIGS. 1-6 that perform the operations described in this application are performed by computing hardware, for example, by one or more processors or computers, implemented as described above executing instructions or software to perform the operations described in this application that are performed by the methods. For example, a single operation or two or more operations may be performed by a single processor, or two or more processors, or a processor and a controller. One or more operations may be performed by one or more processors, or a processor and a controller, and one or more other operations may be performed by one or more other processors, or another processor and another controller. One or more processors, or a processor and a controller, may perform a single operation, or two or more operations.

Instructions or software to control computing hardware, for example, one or more processors or computers, to implement the hardware components and perform the methods as described above may be written as computer programs, code segments, instructions or any combination thereof, for individually or collectively instructing or configuring the one or more processors or computers to operate as a machine or special-purpose computer to perform the operations that are performed by the hardware components and the methods as described above. In one example, the instructions or software include machine code that is directly executed by the one or more processors or computers, such as machine code produced by a compiler. In another example, the instructions or software includes higher-level code that is executed by the one or more processors or computer using an interpreter. The instructions or software may be written using any programming language based on the block diagrams and the flow charts illustrated in the drawings and the corresponding descriptions in the specification, which disclose algorithms for performing the operations that are performed by the hardware components and the methods as described above.

The instructions or software to control computing hardware, for example, one or more processors or computers, to implement the hardware components and perform the methods as described above, and any associated data, data files, and data structures, may be recorded, stored, or fixed in or on one or more non-transitory computer-readable storage media. Examples of a non-transitory computer-readable storage medium include read-only memory (ROM), random-access memory (RAM), flash memory, CD-ROMs, CD-Rs, CD+Rs, CD-RWs, CD+RWs, DVD-ROMs, DVD-Rs, DVD+Rs, DVD-RWs, DVD+RWs, DVD-RAMs, BD-ROMs, BD-Rs, BD-R LTHs, BD-Res, magnetic tapes, floppy disks, magneto-optical data storage devices, optical data storage devices, hard disks, solid-state disks, and any other device that is configured to store the instructions or software and any associated data, data files, and data structures in a non-transitory manner and provide the instructions or software and any associated data, data files, and data structures to one or more processors or computers so that the one or more processors or computers can execute the instructions. In one example, the instructions or software and any associated data, data files, and data structures are distributed over network-coupled computer systems so that the instructions and software and any associated data, data files, and data structures are stored, accessed, and executed in a distributed fashion by the one or more processors or computers.

While this disclosure includes specific examples, it will be apparent after an understanding of the disclosure of this application that various changes in form and details may be made in these examples without departing from the spirit and scope of the claims and their equivalents. The examples described herein are to be considered in a descriptive sense only, and not for purposes of limitation. Descriptions of features or aspects in each example are to be considered as being applicable to similar features or aspects in other examples. Suitable results may be achieved if the described techniques are performed in a different order, and/or if components in a described system, architecture, device, or circuit are combined in a different manner, and/or replaced or supplemented by other components or their equivalents. Therefore, the scope of the disclosure is defined not by the detailed description, but by the claims and their equivalents, and all variations within the scope of the claims and their equivalents are to be construed as being included in the disclosure.

What is claimed is:

1. A blood pressure measurement method, comprising:
receiving an image comprising a skin image of a user;
converting each color data of skin regions of interest in a predetermined part of the skin image into frequency domain data;
calculating a maximum peak value of pulses in pulse related frequency domain data of the frequency domain data, a maximum frequency value corresponding to the maximum peak value, and a maximum peak frequency phase difference of a phase difference of the maximum frequency value between the skin regions;
calculating a pulse transit time as a function of the maximum frequency value and the maximum peak frequency phase difference; and
calculating a blood pressure of the user as a function of the maximum peak value, the maximum frequency value, and the pulse transit time.

2. The method of claim 1, wherein the calculating of the blood pressure comprises receiving a height and a weight of the user, and calculating the blood pressure of the user as a function of the maximum peak value, the maximum frequency value, the pulse transit time, the height, and the weight.

3. The method of claim 1, wherein the predetermined part comprises a plurality of predetermined parts, and the method further comprises:
converting the color data into the frequency domain data for each of the plurality of parts,
calculating the maximum peak frequency phase difference for the frequency domain data for each of the plurality of parts, and calculating the blood pressure for the maximum peak frequency phase difference for the frequency domain data for each of the plurality of parts, and
averaging the calculated blood pressure for the plurality of parts.

4. The method of claim 1, further comprising:
calculating a modified blood pressure of the user using a blood pressure regression analysis equation based on a plurality of first blood pressure data of the calculated blood pressure and a plurality of second blood pressure data measured by a sphygmomanometer.

5. The method of claim 1, wherein the converting of the color data into the frequency domain data comprises:
changing a color system of a plurality of pixels of the skin image corresponding to each of the skin regions from an RGB color system to YC_{g}Cₒ and YC_{b}Cᵣ color systems;
calculating a weighted average value of C_{g} and C_{b} color data included in the YC_{g}Cₒ and YC_{b}Cᵣ color systems; and
applying Fast Fourier Transform (FFT) to the weighted average value of the C_{g} and C_{b} color data.

6. A blood pressure measurement apparatus, comprising:
an interface configured to receive a captured image including a skin image of a user; and
one or more processors configured to
convert each color data of skin regions of interest in a predetermined part of the skin image into frequency domain data,
calculate a maximum peak value of pulses in pulse related frequency domain data of the frequency domain data, a maximum frequency value corresponding to the maximum peak value, and a maximum peak frequency phase difference of a phase difference of the maximum frequency value between the skin regions,
calculate a pulse transit time as a function of the maximum frequency value and the maximum peak frequency phase difference, and
calculate the blood pressure of the user as a function of the maximum peak value, the maximum frequency value, and the pulse transit time.

7. The blood pressure measurement apparatus of claim 6, wherein when a height and a weight of the user are further received through the interface, the one or more processors are further configured to calculate the blood pressure of the user as a function of the maximum peak value, the maximum frequency value, the pulse transit time, the height, and the weight.

8. The blood pressure measurement apparatus of claim 6, wherein when there is a plurality of predetermined parts of the skin image, the one or more processors are configured to calculate the blood pressure for each of the plurality of parts, and an average blood pressure of the blood pressure for each of the plurality of parts.

9. The blood pressure measurement apparatus of claim 6, wherein the one or more processors are configured to further calculate a modified blood pressure of the user using a blood pressure regression analysis equation based on a plurality of first blood pressure data of the calculated blood pressure and a plurality of second blood pressure data measured by a sphygmomanometer.

10. The blood pressure measurement apparatus of claim 6, wherein the one or more processors are configured to change a color system of a plurality of pixels of the plurality of images included in the image corresponding to the skin regions from an RGB color system to YC_{g}Cₒ and YC_{b}Cᵣ color systems, calculate a weighted average value of C_{g} and C_{b} color data included in the YC_{g}Cₒ and YC_{b}Cᵣ color systems, and apply Fast Fourier Transform (FFT) to the weighted average value of the C_{g} and C_{b} color data.

11. A blood pressure measurement method, comprising:
receiving an image comprising a skin image of a user;
converting color data of skin regions of interest in one or more predetermined parts of the skin image into corresponding frequency domain data;
calculating maximum peak values of pulses in pulse related frequency domain data of the corresponding frequency domain data, maximum frequency values corresponding to the maximum peak values, and maximum peak frequency phase differences of the maximum frequency values;
calculating pulse transit times of corresponding ones of the maximum frequency values and the maximum peak frequency phase differences; and
calculating blood pressures as a function of corresponding ones of the maximum peak values, the maximum frequency values, and the pulse transit times.

12. The method of claim 11, where an average blood pressure of the blood pressures is indicated as a blood pressure of the user.

13. The method of claim 12, wherein the function of calculating of the blood pressure further comprises a height and a weight of the user.
